(19) United States
(12) Patent Application Publication
(10) Pub. No.: US 2002/0018806 A1
(43) Pub. Date: Feb. 14, 2002

(54) Lipopeptide adjuvants

(76) Inventors: Babita Agrawal, Edmonton (CA); Michael B. Longenecker, Edmonton (CA); Joanne Parker, Edmonton (CA)

Correspondence Address: Bernhard D. Saxe, FOLEY & LARDNER, Washington Harbour, 3000 K Street, N.W., Suite 500, Washington, DC, 20007-5109, US

(21) Appl. No.: 09/815,346

(22) Filed: Mar. 23, 2001

Related U.S. Application Data
(60) Provisional application No. 60/191,736, filed on Mar. 24, 2000.

(51) Int. Cl. A61K 9/127; A61K 39/12; A61K 39/02; A61K 39/00

(52) U.S. Cl. 424/450; 424/204.1; 424/277.1; 424/234.1

(57) ABSTRACT

Vaccine compositions containing a MUC-1-based adjuvant and an antigen are useful in treating and preventing disorders such as cancer and viral diseases. Exemplary compositions contain a 25-amino acid lipopeptide adjuvant and an antigen of interest in association with a liposome.

### BACKGROUND OF THE INVENTION

Immunotherapy or vaccine therapy approach is an attractive form of therapy for certain viral, bacterial infections and various cancers. However, immunotherapy for these diseases is restricted partially due to the fact that a number of target antigens (peptides, glycopeptides, lipids, lipopeptides, carbohydrates etc.) are poorly immunogenic or induce non-desirable type of immune responses, e.g., antibody response only or type 2 T cell responses only. This specific skew in immune response towards a specific antigen is in part dependent upon the major histocompatibility complex molecules, in vivo environment, pre-exposure to another infection and T cell repertoire etc.

An ideal vaccine antigen should contain both B and T cell epitopes. An effective immune response would consist of both antibody and cytotoxic T cell mediated effector functions. Generation of both antibody and cytotoxic T cell responses against a given antigen requires that a strong T helper cell response is generated. T helper cell responses are provided by CD4+ T cells that recognize fragments of peptide antigens in context of MHC class II molecules on the surface of antigen presenting cells (APCs). Most of the processed forms of peptide antigens are only able to be presented by one or a few alleles of MHC haplotypes. Therefore, T helper response to a given antigenic peptide becomes strictly under control of genetic makeup of an individual. Therefore, inclusion of a helper epitope in most cases would become restricted to one or a few restricted haplotypes of MHC out of a divergent population with highly polymorphic MHC molecules. This genetically restricted T helper cell stimulatory activity of peptide antigens presents a serious obstacle and consequently such T helper epitopes become of limited practical value as a vaccine candidate for majority of an outbred population.

In order to avoid the above limitation with T helper peptide epitopes, large proteins have been utilized as carrier molecules. However, use of large proteins as carriers is expensive, variable and may result in adverse effects upon repeated administrations.

Therefore, identification of T helper epitope peptides that can be presented in context of a vast majority of haplotypes of MHC class II molecules and therefore induce strong CD4+ T helper responses in majority of outbred human population, is highly desirable. Such T helper peptide epitopes are generally referred to as “Promiscuous” or “Permissive”T helper epitopes. Such promiscuous T helper epitopes have been defined and identified before, e.g., tetanus toxoid peptide, Plasmodium falciparum (pfg27), Lactate dehydrogenase, HlVgp120 etc. (Infect. Immun, 1998; 66:3579-3590, C E Contreas et al; J. A.I.D.S. Human Retrovirol 1997; 14:91-101, P. Gaudebout et al; J. Mol. Recog. 1993; 6:81-94, P T Kaumaya et al; J. Immunol.1992;148:907-913, J. Fern and M F Good).

Some of these promiscuous T helper epitopes have also been shown in conjunction with other antigens to induce strong B cells response to a given antigen as well as to bypass certain haplotype restricted immune responses (J. Mol. Recog., 1993, 6:81-94, P T Kaumaya et al).

A need exists in the art, therefore, for promiscuous epitopes useful in enhancing and generalizing the immune response against otherwise inferior antigens.

### SUMMARY OF THE INVENTION

It is an object of the invention to provide compositions and methods that overcome the deficiencies of the art.

According to this object, the invention provides a vaccine composition, containing a MUC-1-based adjuvant peptide and an antigen. In one aspect, the adjuvant is from about 12 to about 25 amino acids long, yet in other it is from about 9 to about 11 amino acids long. The adjuvant may be lipid or carbohydrate modified. In addition, the adjuvant and antigen may be covalently linked or part of a fusion protein. Possible antigens, which also may be lipid-modified, include viral antigens, tumor antigens, parasite antigens and bacterial antigens. In a preferred aspect, the vaccine contains a liposome.

Also according to this object, the invention provides a method of stimulating the immune response of a patient. In one embodiment, the method involves administering to a patient an inventive vaccine. In an alternative embodiment, the method entails contacting ex vivo a T-cell and/or and APC from a patient with an inventive vaccine and administering T-cell and/or an APC to the patient.

### DETAILED DESCRIPTION OF THE PREFERRED EMBODIMENTS

We have identified a promiscuous T helper epitope from the peptide sequence of extracellular tandem repeat domain of MUC1 mucin. This promiscuous T helper epitope could be used therapeutically in conjunction with other poorly immunogenic or non-immunogenic antigens to induce strong immune responses. This epitope could also be used to bypass MHC haplotype restriction for certain antigens.

Accordingly, the invention relates to vaccine compositions and their use in stimulating a patient's immune system. The present vaccines have two basic components: a promiscuous MUC-1-derived T-cell antigen (and “adjuvant” for the purposes of the invention) and a non-MUC-1-antigen. The promiscuous MIUC-1 -derived antigen acts as an adjuvant to generate or enhance an immune response to the antigen upon administration to a patient.

Because the inventive vaccine compositions incorporate a “promiscuous” or “permissive” T-cell antigen derived from MUC-1, they are particularly effective at generating an immune response to an antigen against which the patient otherwise would not respond or would not respond to therapeutically or prophylactically effective levels.

As used herein with reference to MUC-1 -derived peptides, “promiscuous” and “permissive” are used interchangeably to indicate a general lack of specificity for any particular HLA molecule. Such a peptide may bind to class I or class II molecules and among the different subclasses of class I and class II molecules. The skilled artisan will be familiar with assays for measuring promiscuity. These promiscuous MUC-1-derived peptides are also referred to herein as “adjuvants.”

The promiscuous MUC-1-derived peptides useful in the present invention are used in conjunction with a target antigen molecule, which is a non-MUC-1-antigen. This target antigen can be from any source against which immunity is sought. Due to their general stimulatory character, the promiscuous MUC-1-derived peptides are useful adjuvants in generating or enhancing an immune response against the target antigen.

Promiscuous MUC-1-Derived Peptides (Adjuvants)

The promiscuous MUC-1 -derived peptides (adjuvants) are based on the following amino acid sequence: STAPPAHGVTSAPDTRAPGSTAPP. This core region may also be modified to generate “derivatives,” as described in detail below, in ways which the derivative retains the promiscuous nature of the molecule. For example, it may be shorted from the C-terminus to about 12 amino acids and promiscuity should be retained. The basic sequence also may be shorted to about 9 amino acids from the C-terminus and promiscuity among class I molecules should be retained, however, such molecules are expected to lose class II binding capability. Thus, derivatives from about 12 to about 24 amino acids are preferred, because they stimulate both class I and class II molecules, with about 15 to about 20 amino acids providing a quite suitable range. On the other hand, where only class I-associated immunostimulation is desired (e.g., CTL function), it may be desirable to utilize adjuvant molecules having from about 9 to about 11 amino acids. In addition, the following adjuvant “derivatives” are contemplated.

The basic sequence above represents slightly more than a single direct repeat (of up to about a hundred) from the native MUC-1 molecule. Thus, while the sequence is presented as beginning with STAPP, and such molecules are preferred, the invention also contemplates other permutations, beginning at other amino acids, but falling within the size parameters outlined herein. For example, with reference to the above core sequence, molecules could begin TAPPA, APPAH, PPAHG, and so on.

Moreover, one or more amino acids of the core sequence may be altered, preferably in a conservative manner known in the art, such that the requisite promiscuity is maintained, or even enhanced. Typical substitutions may be made among the following groups of amino acids: (a) G, A, V, L and I; (b) G and P; (c) S, C, T, M; (d) F, Y, and W; (e) H, K and R; and (f) D, E, N, and Q. Some preferred substitutions may be made among the following groups: (i) S and T; (ii) P and G; and (iii) A, V, L and I.

Preferred adjuvants are modified with at least one lipid molecule. Exemplary lipid moieties include, but are not limited to, palmitoyl, myristoyl, stearoyl and decanoyl groups or, more generally, any C₂ to C₃₀ saturated, monounsaturated or polyunsaturated fatty acyl group. The serine residues within the MUC I core sequence offer convenient sites where lipid molecules can be attached. An example of such an adjuvant is (1) BP1-217 with two myristyl lipids attached to two serines at the carboxy terminus of the core peptidic sequence; (2) BP1-228 with only one myristyl lipid attached to a carboxy terminal serine;or MUC I peptide, (3) BP1-132 with two palmitate lipid molecules attached to two adjacent carboxy terminal lysine amino acid residue; or (4) BPI-148 with one palmitate lipid molecule attached to a carboxy terminal lysine amino acid residue.

BP1-217: GVTSAPDTRPAPGSTAS(myristyl)S(myristyl)L

BP1-228: GVTSAPDTRPAPGSTAS(myristyl)L

BP1-132: TAPPAHGVTSAPDTRPAPGSTAPPK(palmitate)K(palmitate)G

BPI-148 STAPPAHGVTSAPDTRPAPGSTAPP-Lys(Palmitate)

Adjuvants also may be glycosylated, partially glycosylated, or attached to a carbohydrate according to methods known in the art or modified with large molecular weight polymers, such as polyethylene glycols. An example of such an adjuvant is BPI-216 glycolipopeptide. BPI-216 has two myristyl lipids attached to two serines at the carboxy terminus of the peptide sequence and a Tn carbohydrate O-linked to threonine and serine of the peptide at the GVTS sequence of the MUC1 tandem repeat. Tn carbohydrate antigen is found on a variety epithelial cells derived form adenocarcinomas of the breast, colon, pancreas. It is also associated with Tcell Lymphomas.

BP1-216 GVT(Tn)S(Tn)APDTRPAPGSTAS(Myristyl)S(Myristyl)L

For convenience in making chemical modifications, it is sometimes useful to include in a MUC-1 peptide one or more amino acids having a side chain amenable to modification. A preferred amino acid is lysine, which may readily be modified at the ε-amino group. Side chain carboxyls of aspartate and glutamate are readily modified, as are serine, threonine and tyrosine hydroxyl groups, the cystine sulfhydryl group and the histidine amino group. Such additional amino acids are not included within the size parameters provided above. Thus, while MUC-1 derived peptides may be, for example, from about 12 to about 24 amino acids, the addition of a lysine would alter the size range from about 13 to about 25 amino acids. Likewise, the addition to two modifiable amino acids to the molecules ranging from about 15 to about 20 amino acids would give a range of from about 17 to about 22 amino acids, and so on.

Antigens

The present vaccines apply generally to a great variety of antigens, which may be of nearly any chemical constitution. Exemplary antigens can be derived from peptides, carbohydrates, lipids and especially combinations thereof. Particularly important antigens are peptides, lipopeptides and glycopeptides. Idiotypic and antiidiotypic antigens are specifically included. MUC-1 antigens are not included in the present usage of the term. Lipid-modified peptide antigens (lipopeptide antigens) are a preferred type of antigen.

Antigens against which it would be highly advantageous to use the subject vaccines include tumor antigens. Tumor antigens are usually native or foreign antigens which are correlated with the presence of a tumor. Inasmuch as tumor antigens are useful in differentiating abnormal from normal tissue, they are useful as a target for therapeutic intervention.

Tumor antigens are well known in the art. Indeed, several examples are well-characterized and are currently the focus of great interest in the generation of tumor-specific therapies. Non-limiting examples of tumor antigens are carcinoembryonic antigen (CEA), prostate specific antigen (PSA), melanoma antigens (MAGE, BAGE, GAGE), and mucins, such as MUC-1.

In another embodiment, the antigen is a parasite-associated antigen, such as an antigen associated with leishmania, malaria, trypanosomiasis, babesiosis, or schistosomiasis. Suitable parasite-associated epitopes include, but are not limited to, the following.

| Parasite | Epitope | References |
|---|---|---|
| Plasmodium Falciparum | (NANP)3 | Good et al. (1986) |
| (Malaria) | | J. Exp. Med. 164:655 |
| | Circumsporoz. | Good et al. (1987) |
| | Protein | Science 235:1059 |
| | AA 326-343 | |
| Leishmania donovani | Repetitive peptide | Liew et al. (1990) |
| | | J. Exp. Med. 172:1359 |
| Leishmani major | EAEEAARLQA | |
| | (code) | |
| Toxoplasma gondii | P30 surface protein | Darcy et al. (1992) |
| | | J. Immunolog. 149:3636 |
| Schistosoma mansoni | Sm-28GST antigen | Wolowxzuk et al. (1991) |
| | | J. Immunol 146:1987 |

In another embodiment, the epitope is a viral epitope, such as an epitope associated with human immunodeficiency virus (HIV), Epstein-Barr virus (EBV), or hepatitis. Suitable viral epitopes include, but are not limited to:

| Virus | Epitope | Reference |
|---|---|---|
| HIV gp120 | V3 loop, 308-331 | Jatsushita, S. et al. (1988) |
| | | J. Viro. 62:2107 |
| HIV GP120 | AA 428-443 | Ratner et al. (1985) |
| | | Nature 313:277 |
| HIV gp120 | AA 112-124 | Berzofsky et al. (1988) |
| | | Nature 334:706 |
| HIV | Reverse transcriptase | Hosmalin et al. (1990) |
| | | PNAS USA 87:2344 |
| Flu | nucleoprotein | Townsend et at. (1986) |
| | AA 335-349, 366-379 | Cell 44:959 |
| Flu | haemagglutinin | Mills et al. (1986) |
| | AA48-66 | J. Exp. Med. 163:1477 |
| Flu | AA111-120 | Hackett et al. (1983) |
| | | J. Exp. Med 158:294 |
| Flu | AA114-131 | Lamb, J. and Green N. (1983) |
| | | Immunology 50:659 |
| Epstein-Barr | LMP43-53 | Thorley-Lawson et al. (1987) |
| | | PNAS USA 84:5384 |
| Hepatitis B | Surface Ag | Milich et al. (1985) |
| | AA95-109; | J. Immunol. 134:4203 |
| | AA 140-154 | |
| | Pre-S antigen | Milich, et al. (1986) |
| | AA 120-132 | J. Exp. Med. 164:532 |
| Herpes simplex | gD protein | Jayaraman et al. (1993) |
| | AA5-23 | J. Immunol. 151:5777 |
| | gD protein | Wyckoff et al. (1988) |
| | AA241-260 | Immunobiology 177:134 |
| Rabies | glycoprotein | MacFarlan et al. (1984) |
| | AA32-44 | J. Immunol. 133:2748 |

The epitope may also be associated with a bacterial antigen. Suitable epitopes include, but are not limited to:

| Bacteria | Epitope ID | Reference |
|---|---|---|
| Tuberculosis | 65Kd protein | Lamb et al. (1987) |
| | AA112-126 | EMBO J. 6:1245 |
| | AA163-184 | |
| | AA227-243 | |
| | AA242-266 | |
| | AA437-459 | |
| Staphylococcus | nuclease protein | Finnegan et al. (1986) |
| | AA61-80 | J. Exp. Med. 164:897 |
| E. coli | heat stable enterotoxin | Cardenas et al. (1993) |
| | | Infect. Immunity 61:4629 |
| | heat liable enterotoxin | Clements et al. (1986) |
| | | Infect. Immunity 53:685 |
| Shigella sonnei | form I antigen | Formal et al. (1981) |
| | | Infect. Immunity 34:746 |

Vaccine Compositions of the Invention

The inventive compositions may be formulated for administration in a variety of ways. The pharmaceutical compositions of the invention generally contain an immunologically effective amount of an adjuvant and an antigen. Preferably, the adjuvant and antigen are admixed with a pharmaceutically effective vehicle (excipient). In one embodiment, the adjuvant and the antigen are covalently linked to one another. Such linking may be accomplished using methods known to the skilled worker (e.g., production as a fusion protein or linking using chemical linkers).

Guidance in preparing suitable formulations and pharmaceutically effective vehicles, can be found, for example, in REMINGTON'S PHARMACEUTICAL SCIENCES, chapters 83-92, pages 1519-1714 (Mack Publishing Company 1990) (Remington's), which are hereby incorporated by reference.

Preferred vehicles include liposomes. When liposomes are used, conventional vaccine components like Freund's adjuvant, Keyhole Limpet Haemocyanin (“KLH”), Lipid A, monophosphoryl Lipid A (“MPLA”), and the like are optional; the invention specifically contemplates indpendently their presence or absense. For general details on liposomes, see, for example, Remington's at 1691-92. Techniques for preparation of liposomes and the formulation (e.g., encapsulation) of various molecules, including peptides and oligonucleotides, with liposomes are well known to the skilled artisan. Liposomes are microscopic vesicles that consist of one or more lipid bilayers surrounding aqueous compartments. See, generally, Bakker-Woudenberg et al., Eur. J. Clin. Microbiol. Infect. Dis. 12 (Suppl. 1): S61 (1993) and Kim, Drugs 46: 618 (1993). Liposomes are similar in composition to cellular membranes and as a result, liposomes generally can be administered safely and are biodegradable.

Depending on the method of preparation, liposomes may be unilamellar or multilamellar, and can vary in size with diameters ranging from 0.02 μm to greater than 10 μm. A variety of agents can be encapsulated in liposomes. Hydrophobic agents partition in the bilayers and hydrophilic agents partition within the inner aqueous space(s). See, for example, Machy et al., LIPOSOMES IN CELL BIOLOGY AND PHARMACOLOGY (John Libbey 1987), and Ostro et al., American J. Hosp. Pharm. 46: 1576 (1989).

Liposomes can adsorb to virtually any type of cell and then release the encapsulated agent. Alternatively, the liposome fuses with the target cell, whereby the contents of the liposome empty into the target cell. Alternatively, an absorbed liposome may be endocytosed by cells that are phagocytic. Endocytosis is followed by intralysosomal degradation of liposomal lipids and release of the encapsulated agents. Scherphof et al., Ann. N.Y. Acad. Sci. 446: 368 (1985). Irrespective of the mechanism or delivery, however, the result is the intracellular disposition of the associated therapeutic.

Anionic liposomal vectors have also been examined. These include pH sensitive liposomes which disrupt or fuse with the endosomal membrane following endocytosis and endosome acidification.

Among liposome vectors, cationic liposomes are the most studied, due to their effectiveness in mediating mammalian cell transfection in vitro. They are often used for delivery of nucleic acids, but can be used for delivery of other therapeutics, be they drugs or hormones.

Liposomes are preferentially phagocytosed into the reticuloendothelial system. However, the reticuloendothelial system can be circumvented by several methods including saturation with large doses of liposome particles, or selective macrophage inactivation by pharmacological means. Classen et al., Biochim. Biophys. Acta 802: 428 (1984). In addition, incorporation of glycolipid- or polyethylene glycol-derivatised phospholipids into liposome membranes has been shown to result in a significantly reduced uptake by the reticuloendothelial system. Allen et al., Biochim. Biophys. Acta 1068: 133 (1991); Allen et al., Biochim. Biophys. Acta 1150: 9 (1993).

Cationic liposome preparations can be made by conventional methodologies. See, for example, Feigner et al, Proc. Nat'l Acad. Sci USA 84:7413 (1987); Schreier, J. of Liposome Res. 2:145 (1992); Chang et al. (1988), supra. Commercial preparations, such as Lipofectin (Life Technologies, Inc., Gaithersburg, Md. USA), also are available. The amount of liposomes and the amount of DNA can be optimized for each cell type based on a dose response curve. Feigner et al., supra. For some recent reviews on methods employed see Wassef et al., Immunomethods 4: 217-222 (1994) and Weiner, A. L., Immunomethods 4: 217-222 (1994).

Other suitable liposomes that are used in the methods of the invention include multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). The skilled artisan will recognize that the techniques for preparing these liposomes are well known in the art. See COLLOIDAL DRUG DELIVERY SYSTEMS, vol. 66 (J. Kreuter, ed., Marcel Dekker, Inc. 1994).

An example of a liposomal vaccine is BLP25. BLP25 is comprised of a liposomal delivery system, an antigen, and the BPI-148 lipopeptide adjuvant.

Other forms of delivery particle, for example, microspheres and the like, also are contemplated.

Therapeutic and Prophylactic Methods of the Invention

The methods of the invention may be accomplished in vivo or ex vivo. In vivo approaches generally entail administering to a patient an immunogenically effective amount of an inventive vaccine composition. An effective amount is an amount sufficient to enhance a weak immune response to the antigen or an amount sufficient to generate an immune response where, absent the adjuvant, a response could not be generated.

The inventive methods are useful in both therapeutic and prophylatic contexts. Thus, if a patient is suffering from a disorder, the methods may be used to mitigate that suffering. Likewise, used prophylactically (prior to disease onset), the present methods can be used to prevent or lessen the severity of a disorder.

In an ex vivo approach, the inventive vaccines may be used to generate an immune response ex vivo. In particular, immune cells (peripheral blood lymphocytes or isolated dendritic cells, for example) from a patient may be used to prime a patient's T-cells in vitro. In general, antigen presenting cells are loaded with an inventive vaccine composition and the resultant loaded cells are used as antigen presenting cells to generate antigen-specific T-cells, which may then be infused back into a patient in need of treatment. The artisan will be familiar, from the literature, with approaches such as this. The present vaccine compositions can be used in any such method.

The following examples are for illustrative purposes and are not meant to be limiting.

### EXAMPLES

### Example 1

### T Cell Response to BLP25 in Normal Donors

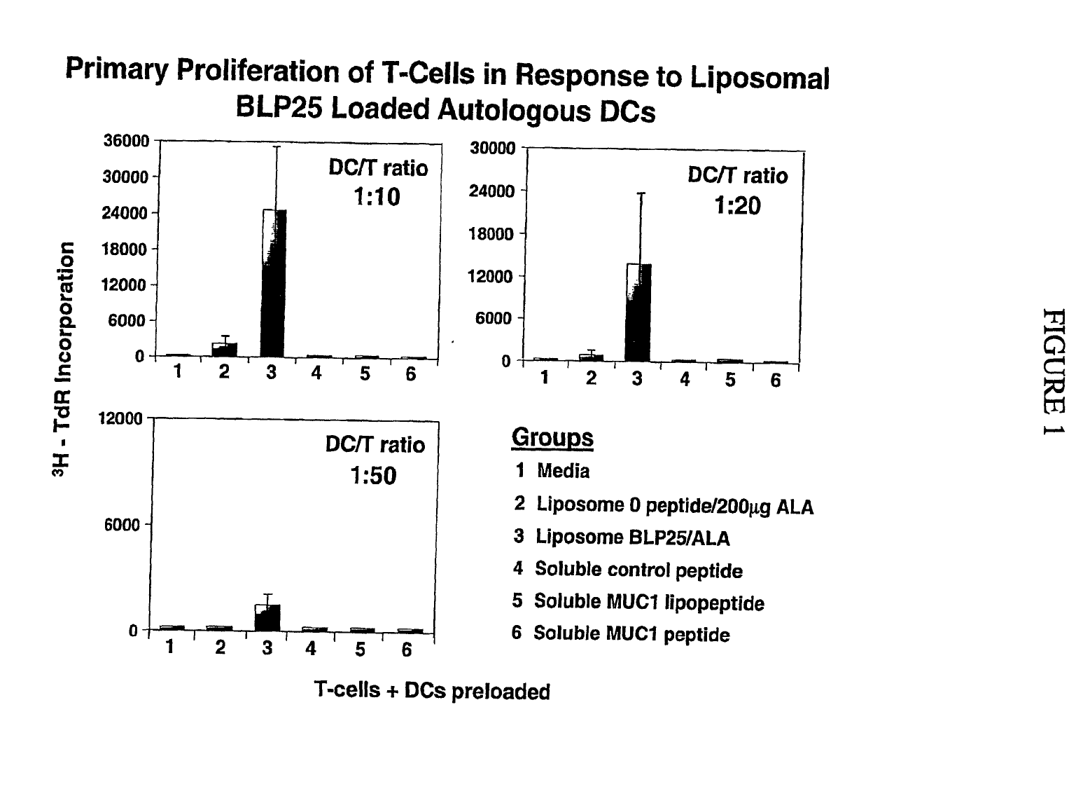

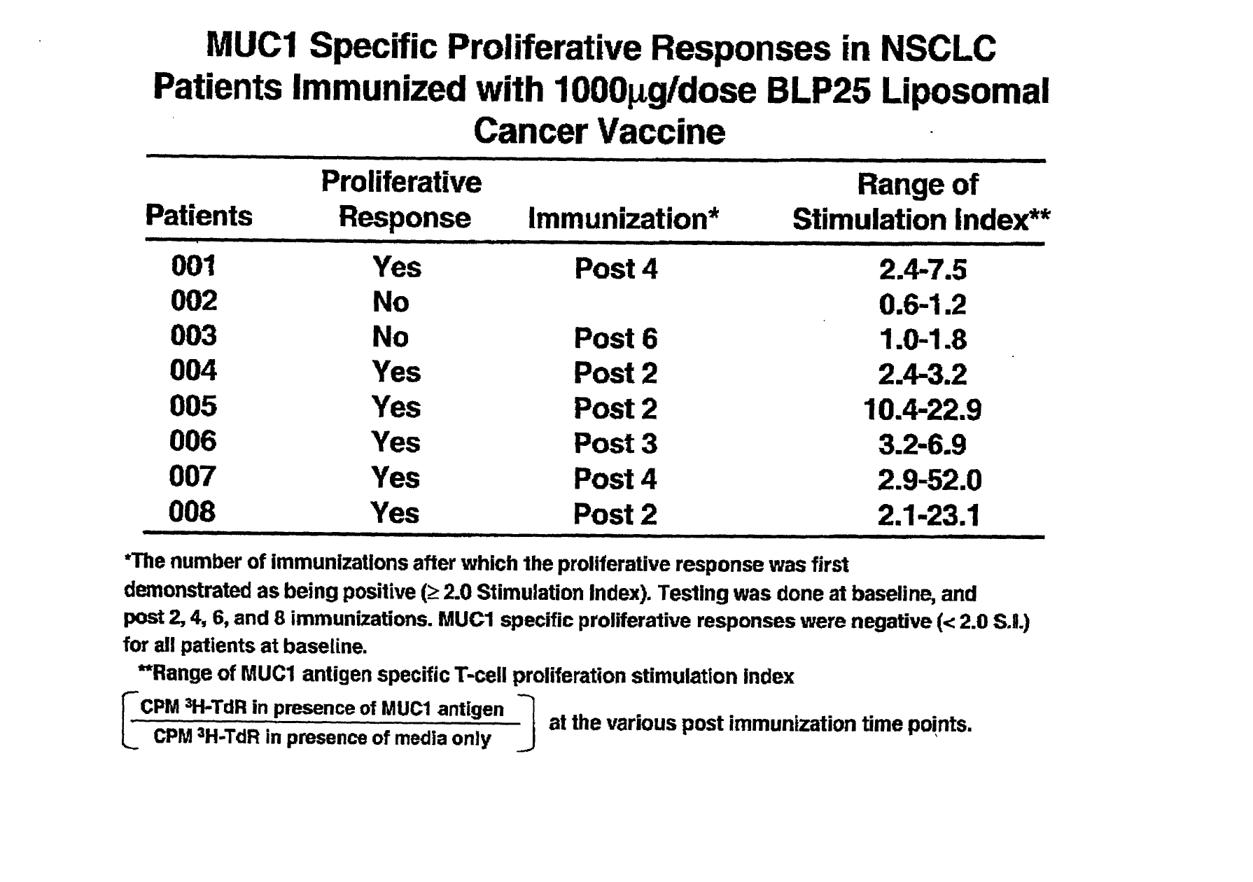

This example demonstrates that BLP25 generates a surprisingly strong immune response, which is suggestive of the promiscuous nature of the antigen. Buffy coats were collected from Canadian Blood Servies from normal donors. Buffy coats were used to purify monocytes (Miltenyi MACS column for CD14+ cells) and T cells (nylon wool columns). The CD14+ monocytes were cultured in presence of GM-CSF (50 ng/ml) and IL-4 (10 ng/ml) for 3 days. At this time, the immature dendritic cells were (DCs) were harvested and further cultured for additional 3 days in presence of media, liposomes containing BLP25 at 400 μg/ml or no antigen and Avanti lipid A. After this culture, the antigen loaded DCs were washed, irradiated and added to autologous T cells for 5-6 days of culture in 96 well flat bottom plates. At this time, the wells were pulsed with 3H-thymidine overnight and 3H-Tdr incorporation into proliferating T cells was determined by counting in a liquid scintillation counter. FIG. 1 represents one experiment out of 6 reproduced experiments (all from different donors). In all of these 6 donors, strong T cell proliferative response was observed suggesting promiscuous nature of BLP25.

### Example 2

### T Cell Proliferative Response of Non Small Cell Lung Cancer (NSCLC) Patients Against BLP25

In a phase II clinical trial, eight NSCLC patients were immunized with liposomal BLP25 vaccine at 1000 ug/injection on a weekly basis for eight weeks. Blood was drawn a week after every two injections and peripheral blood mononuclear cells were separated by FicoII method. Proliferative responses were determined in response to soluble BLP25 in vitro cultures. As indicated in Table I, PBMCs from six out of eight immunized patients showed a strong proliferative response against BLP25. These results further confirm promiscuous T helper nature of BLP25.

### Example 3

### Ascertaining Antigen Promiscuity

In order to determine the adjuvant activity of BLP25, a liposome containing BLP25, a 9 mer telomerase peptide or a glycopeptide antigen are formulated and used to stimulate human T cells in vitro using dendritic cells as efficient antigen presenting cells (APCs). T cell responses are determined against both BLP25 and the telomerase peptide cytotoxic activity as a measure of immune response. An enhancement of the response against telomerase in the presence of BLP25 is indicative of the adjuvant effect.

In general, PCT/US98/09288; Agrawal et al., Int'l Immunol.10:1907-16 (1998); and Agrawal et al., Cancer Res. 55:5151-56 (1 998) provide suitable methods, and those disclosures are hereby incorporated by reference, in their entirety.

Peotides

Telomerase-derived antigenic peptides used in this experiment: RLVDDFLLV, ELLRSFFYV and ILAKFLHWL.

Preparation of Liposomes

The bulk liquid composition of liposomes consist of dipalmitoyl phosphatidyl choline (DPPC), cholesterol (Chol) and dimyristoyl phosphatidyl glycerol (DMPG) in a molar ratio of 3:1:0.25 and contain Lipid A at a concentration of 1% (w/w) of bulk lipid. Synthetic telomerase peptides are present in the aqueous phase during liposome formation at a concentration of 0.7 mg/ml BLP25 also is present, except for a control sample. The formulated product contains 2 mg of bulk lipid, 20 μg Lipid A, with or without about 40 μg BLP25, and about 20 μg of peptide per 100 μl.

General Procedures for Loading APCs with Liposome-encapsulated Peptide

Briefly, to 2−10×10⁶ human dendritic cells in 0.9 mL AIM-V media, one dose of liposome containing peptide formulation is added and the cells were incubated overnight at 37° C. with CO₂ supplemented incubator. After incubation, the cells are treated with mitomycin C or γ-irradiated (3000 rads) followed by washing with AIM-V media.

Cytotoxic T Lymphocyte Assays

For the CTL assay, T-cells are grown for five weeks in bulk cultures. At the end of two weeks, live T-cells are harvested from flasks and counted. The targets are mutant T2 cells. Houbiers et al., Eur. J. Immunol 23:2072-2077 (1993); Stauss et al., Proc. Natl. Acad. Sci. U.S.A. 89:7871-7875 (1992). T2 cells are loaded overnight at 37° C. in 7% CO₂, with or without BLP25, with various the telomerase synthetic peptides at 200 μM in presence of 8 μg exogenous β2 microglobulin. Houbiers et al., supra; Stauss et al., supra. The peptide-loaded T2 target cells are loaded with ⁵¹Cr (using NaCrO₄) for 90 minutes and washed. CTL assays are performed as previously described. Agrawal et al., J. Immunol. 156:2089 (1996). Percent specific killing is calculated as: experimental release−spontaneous release/maximum release−spontaneous release×100. The effector versus target ratios used is 50:1, 25:1, 10:1 and 5:1. Each group is set up in four replicate and mean percent specific killing is calculated.

### Example 4

### Demonstration of T-cell Promiscuity of BPI-148 in Unimmunized Humans

This example demonstrates that BPI-148 generates a strong immune response, which is suggestive of the promiscuous nature of BPI-148. Ficoll-Paque (Pharmacia; Uppsala, Sweden) separated peripheral blood monocyte cells were isolated from the peripheral circulatory system and cultured in AIM V (life Technologies, Gaithersberg, Md.) plus 5% human AB serum at 3×10⁵/well in 4-5 replicates in the presence or absence of BPI-148 or tetanus toxoid lipopeptide for 5-6 days in 96 well flat-bottom plates. At this time, the wells were pulsed with 1 μCi/well ³H-thymidine (Amersham Canada Limited; Oakville, Ontaria) for 18 hours and ³H-Tdr incorporation into DNA was measured after harvesting the cells onto filter and counting in liquid scintillation counter. The results for this experiment are shown below in Table 2 A strong T cell proliferative response was observed suggesting the promiscuous nature of BPI-148.

TABLE 2

| Lipopeptide in culture | *Responder/Total |
|---|---|
| BPI-148 | 10/22 |
| Tetanus toxoid | 10/21 |

Responders are defined as peripheral blood mononuclear cells giving ≧2 S.I. (S.I.=counts per minute in the presence of antigen/counts per minute in the absence of antigen, media only).

What is claimed is:

1. A vaccine composition, comprising a MUC-1-based adjuvant peptide and an antigen.

2. A vaccine according to claim 1, wherein said adjuvant is from about 12 to about 25 amino acids long.

3. A vaccine according to claim 1, wherein said adjuvant is from about 9 to about 11 amino acids long.

4. A vaccine according to claim 2, wherein said adjuvant is lipid-modified.

5. A vaccine according to claim 2, wherein the adjuvant is BPI-217 or a derivative thereof.

6. A vaccine according to claim 2, wherein the adjuvant is BPI-228 or a derivative thereof.

7. A vaccine according to claim 2, wherein the adjuvant is BPI-132 or a derivative thereof.

8. A vaccine according to claim 2, wherein the adjuvant is BPI-148 or a derivative thereof.

9. A vaccine according to claim 2, wherein the adjuvant is BPI-216 or a derivative thereof.

10. A vaccine according to claim 1, wherein said antigen is selected from the group consisting of viral antigens, tumor antigens, parasite antigens and bacterial antigens.

11. A vaccine according to claim 1, wherein said antigen is lipid-modified.

12. A vaccine according to claim 11, wherein said antigen is a selected from the group consisting of viral antigens, tumor antigens, parasite antigens and bacterial antigens.

13. A vaccine according to claim 1, further comprising a delivery vehicle.

14. A vaccine according to claim 13, wherein said delivery vehicle is a liposome.

15. A vaccine according to claim 1, wherein said adjuvant and said antigen are covalently linked to one another.

16. A method of stimulating the immune response of a patient, comprising administering to said patient the vaccine of claim 1.

17. A method of stimulating the immune system of a patient, comprising contacting ex vivo a T-cell from the patient with the vaccine of claim 1 and administering to the patient the contacted cells.
